(19) United States
(12) Patent Application Publication
(10) Pub. No.: US 2006/0089610 A1
(43) Pub. Date: Apr. 27, 2006

(54) Skin-peeling pad

(76) Inventor: Norio Daikuzono, Tokyo (JP)

Correspondence Address: KIRK HAHN, 14431 HOLT AVE, SANTA ANA, CA, 92705, US

(21) Appl. No.: 10/974,374

(22) Filed: Oct. 27, 2004

(51) Int. Cl. A61M 35/00

(52) U.S. Cl. 604/289

(57) ABSTRACT

A method for the safe, easy and inexpensive peeling of the skin with simultaneous release of a medicinal or cosmetical substance to penetrate the skin without subjecting the recipient to pain.

### BACKGROUND OF THE INVENTION

The present invention relates to a technology that causes a medicinal, cosmetical or other substance to be absorbed into the skin and thus permeate it, as well as, a peeling pad suitable for the implementation of such technology.

A barrier formed on the skin surface by the waste byproducts of the skin, stratum corneum (i.e. dead skin cells) and other tissues is known to be a factor that inhibits the permeation of substances into the skin. Medicinal, cosmetical, or other substances, when applied to the skin surface, are inhibited by this barrier from absorbing through the skin surface and permeating into the living body's internal tissues. Peeling away this skin-surface barrier, before applying the effective substance to the skin's surface, is a way to aid permeation.

The acknowledged peeling methods include: peeling using tape; the use of a chemical agent such as hyaluronic acid; a sandblaster or other abrasive method in which particles are sprayed at a high velocity onto the surface of the skin; and transpiration of the living body's surface tissues using a CO₂ laser, erbium YAG laser, etc. Additionally, in the field of cosmetics, inorganic particles ranging in size from several microns to several tens of microns can be applied to the skin's surface and massaged by hand to remove the aforementioned waste byproduct and stratum corneum can be used to tone or clean the skin's surface. A typical method is described in Japan Patent 56-131512 by Toku-Kai-Sho.

Among the conventional peeling methods mentioned above, peeling using tape is the most primitive method but can be implemented with little cost or effort. However, this method cannot peel the skin surface barrier of several tens of microns to a suitable degree of uniformity or precision. Moreover, the process can be painful for the recipient of such treatment.

The transpiration of the skin-surface tissues using laser can peel the skin surface barrier uniformly and precisely, but the implementation of such a method requires an expensive system.

Abrasion is not able to peel away the skin's surface barrier at the required degree of uniformity and precision, and an expensive system is required to implement the method.

Compared with the above methods, a method of peeling the skin using a cream or gel containing inorganic particles, such as an abrasive compound, is desirable because it is inexpensive and easy to use without subjecting the recipient to much pain. However, a large number of particles will remain on the face as the skin is peeled, and such particles may enter the eyes or mouth to cause ocular damage or leave the sensation of a foreign body in the mouth. Additionally, the skin must be washed after peeling to rinse away any particles left on the skin's surface. Therefore, even when a cream or gel containing a medicinal or cosmetical substance is applied to the skin during peeling, most of the cream or gel will be removed when the skin is washed, whereupon the effect of permeating the effective substance into the skin drops substantially. Because the particles remain on the skin, this method must be implemented in three steps of peeling, washing, and application and permeation of the effective substance. In other words, the peeling of the skin and the permeation of a medicinal or chemical substance into the skin cannot be performed simultaneously. As a result, the treatment becomes complex and costly, meaning it is not suitable for home care or other applications requiring simplicity and safety. Furthermore, the crushed grains have sharp edges and are unstable in shape. If these grains penetrate through the skin barrier of several hundred microns or the stratum corneum layer of several millimeters in maximum thickness, the skin or eyes can be pierced, possibly resulting in skin or ocular damage or bacterial infection.

Accordingly, the main challenge of the present invention is to provide a technology to peel the skin safely, inexpensively and easily while causing a medicinal or cosmetical substance to penetrate the skin at the same time, without subjecting the recipient to pain.

### BRIEF SUMMARY OF THE INVENTION

The present invention solves the above-mentioned problems in the manner explained below:

Specifically, the first embodiment provides a method of peeling the skin's surface using a low-hardness skin-peeling pad whose surface contains a large number of non-needle shaped particles averaging less than 1 micron in size, while causing simultaneously a medicinal or cosmetical substance to penetrate the skin.

The skin will be peeled only to a limited depth, within the particle size range, when applied and massaged with the peeling pad because the pad (holding the non-needle shaped particles that contact the skin) is soft and has a low hardness (equivalent to or less than the hardness of the skin), and also because the grains are not needle shaped. Although the depth of such peeling will change according to the particle size, pad hardness and pressure level, it will not exceed the particle size as mentioned above. For example, when a thick stratum corneum layer of 1 mm or more needs peeling, the use of a peeling pad with an average particle size of 1 mm will not peel the skin beyond the 1 mm thickness of the stratum corneum layer. Since the grains are not needle shaped, the chances of such particles piercing the skin or eyes and causing bacterial infection are minimal. This is even true when the particles remain on the surface of the skin by mistake due to them separating from the pad from a manufacturing defect or poor performance of the holding binder.

The materials suitable for use as particles include glass, quartz, ceramic, polycarbonate, acrylic resin, PET or other hard plastic material having a hardness sufficient to peel the skin.

The second embodiment provides a skin-peeling pad, in which the particles are held, made of a continuously formed material. This continuously formed material can absorb or adsorb an effective agent in powder, liquid, cream or gel form that contains a medicinal or cosmetical substance. The use of this peeling pad, into or onto which an effective agent is absorbed or adsorbed, will effectively reduce the time and complexity of treatment because the number of times the agent is applied to the pad can be reduced. Additionally, the peeling pad into or onto which an effective agent is absorbed or adsorbed can be made disposable, facilitating better hygiene and consequently safer treatment.

The third embodiment has non-needle shaped particles with numerous pores adsorbed onto skin-peeling pad. Non-needle shaped particles, having many pores of 1 micron or less in diameter, as well as, a specific surface area of 100 m² or more per gram, can absorb or adsorb an effective agent in powder, liquid, cream or gel form that contains a medicinal or cosmetical substance. The skin-peeling pad provided by the present invention does not require the application of an effective agent in powder, liquid, cream or gel form that contains a medicinal or cosmetical substance, but it allows such medicinal, cosmetical or other substance selectively to permeate the skin in a simpler, more hygienic manner during peeling. As is the case with the second invention, the peeling pad herein described can be made disposable, facilitating better hygienic and consequently safer treatment.

The fourth invention provides a handle on which a skin-peeling pad is placed. A peeling pad affixed to a hard holder is set onto the handle. When this handle is used to massage the skin, more uniform pressure can be applied to the skin, resulting in more uniform peeling and safer treatment.

### BRIEF DESCRIPTIONS OF THE DRAWINGS

FIG. 1 illustrates a longitudinal section through the handle of an embodiment of the invention showing the pad holder and peeling pad detached from the handle.

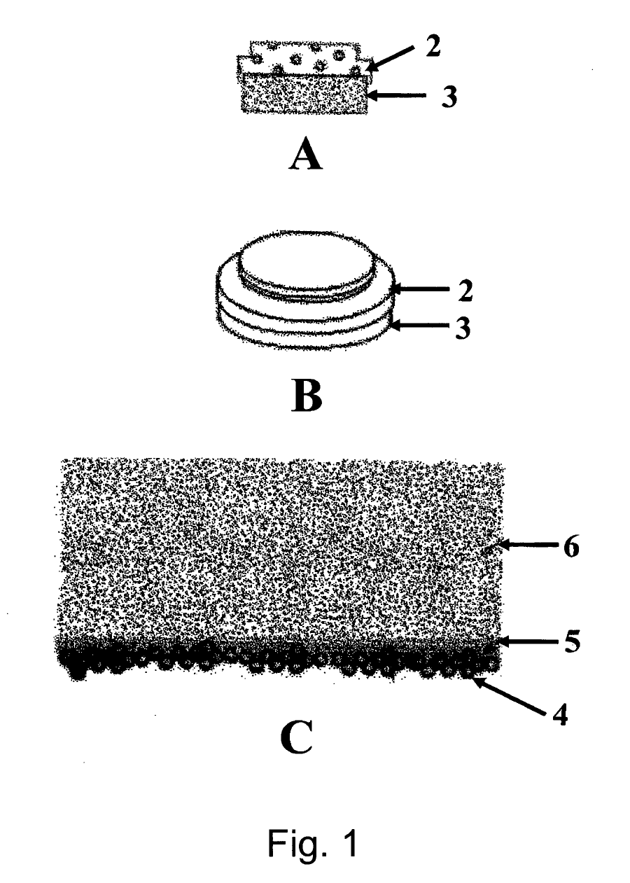

FIG. 2 illustrates a longitudinal section through the handle of an embodiment of the invention showing the pad holder and peeling pad attached to the handle.

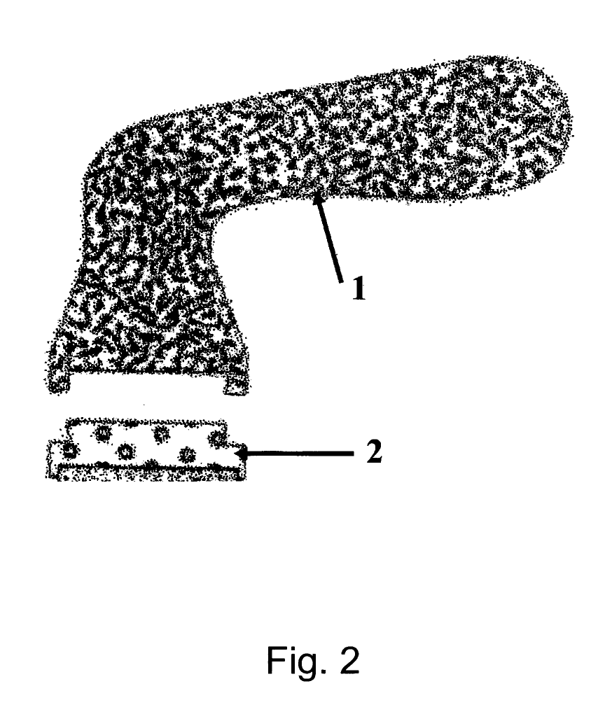

FIG. 3 illustrates: A. a longitudinal section through the pad holder and peeling pad, B. a three-dimensional perspective of the top of the pad holder, and C. a detailed drawing of a cross-section through the peeling pad.

### EXPLANATION NUMBERS IN DRAWING

(1) Handle

(2) Pad holder

(3) Peeling pad

(4) Non-needle shaped particle

(5) Adhesive or binder for affixing or holding non-needle shaped particles on the pad

(6) Pad

### DETAILED DESCRIPTION OF THE INVENTION

A detailed description of the instant invention is presented below:

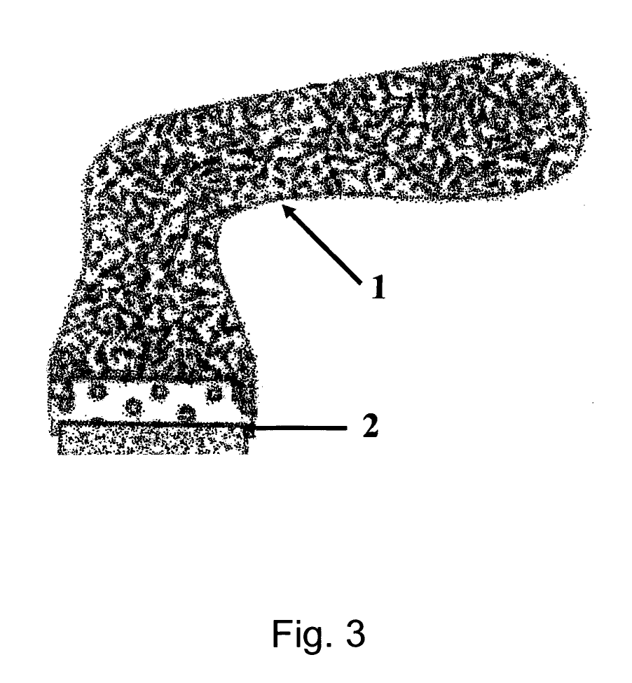

As shown in FIG. 3, the non-needle shaped particles (4) with have a ratio of longest axis to shortest axis of 1:0.5 to 1:1 or round, as specified by the present invention, should preferably be quartz or sapphire, which are inorganic and cause minimal reaction in the living body. However, it is also possible to use polycarbonate, acrylic resin or PET materials whose hardness is sufficiently greater than that of the skin.

The non-needle shaped particles should preferably be truly spherical in shape, but non-needle shaped particles of near-spherical shape having no projections can be used as the particles because they will not pierce the skin.

The material for the peeling pad (6) shown in FIG. 3 should ideally be silicone, polyurethane, polyethylene, natural rubber or other continuously formed material that can amply absorb an effective agent in powder, liquid, cream or gel form that contains a medicinal or cosmetical substance. However, a non-foamed material of low hardness can also be used, although the effective agent must be applied more frequently. The pad should preferably be a disc at least 2 mm thick and 20 mm or more in diameter, but the pad's surface that contacts the skin can have any shape, being circular, oval or diamond.

As shown in FIG. 3, the non-needle shaped particles averaging 1 mm or less in size, as specified by the present invention, are affixed or held onto the surface of the low-hardness pad via an adhesive layer or binder layer (5). A liquid adhesive of the same material or a liquid that dissolves the pad is applied to the pad's surface, and then the pad is pressed onto the non-needle shaped particle powder to securely adhere and affix the non-needle shaped particles. Once the adhesive or binder layer has solidified, the excess particles that have not yet adhered are removed. If a thermoplastic pad material is used, the particles can also be affixed or held onto the pad's surface by pressing the pad surface onto the non-needle shaped particles to adhere them while the pad or its surface is subjected to heat-melting.

The solidified adhesive or binder should have a low hardness of JIS 30 degrees or a Shore hardness 30 A or less, just like the pad material. This way the entire peeling pad will have a flexible structure, so that the pad's entire surface will contact the skin with uniform pressure and allow for more uniform peeling of the skin, which is complex in shape.

The effective agent containing a medicinal or cosmetical substance for permeation into the skin may be a soap or shampoo in cream, gel, emulsion, lotion, liquid or solid form.

The non-needle shaped particles having numerous pores of 1 micron or less in size, as well as a BET specific surface area of 100 m² or more per gram, can selectively absorb or adsorb a medicinal or cosmetical substance of different particle size when the pore diameter is changed. A non-needle shaped particle having many pores has a large surface area. Therefore, when these particles are adhered to the pad, the adhesion area becomes large and the adhesive strength of the peeling pad, and ultimately its durability and safety, will improve.

A peeling pad that holds non-needle shaped particles having no pores requires that the effective agent containing a medicinal or cosmetical substance be applied to the pad to cause the agent to penetrate the skin. By contrast, a peeling pad that holds non-needle shaped particles with many pores can absorb the medicinal or cosmetical substance into the pores, or adsorb the substance onto the surface. Then, upon contact with the skin, the substance is released from the particles onto the peeled skin and dissolves in the moisture or oil content of the skin. It is also possible to cause the medicinal or chemical substance to be released rapidly from the pores. By causing hydrophilic non-needle shaped particles having pores to absorb or adsorb an oil-based medicinal or cosmetical substance into their pores beforehand, the absorbed/adsorbed substance can be released rapidly just by applying water to the skin or the peeling pad before treatment. Alternatively, by causing lipophilic non-needle shaped particles having pores to absorb or adsorb a water-based medicinal or cosmetical substance into their pores beforehand, the absorbed/adsorbed substance can be released rapidly just by applying mineral oil, etc., to the skin or the peeling pad before treatment. The medicinal or cosmetical substance thus released permeates the skin as the skin is peeled.

If the skin-peeling pad (3) is to be placed on a handle (1) as shown in FIG. 3, the peeling pad should preferably have a structure that permits detachment from the handle. Since the peeling pad is affixed to a resin pad holder (2) having high hardness, it is preferable that the entire pad holder, with pad, be “attachable to”/“detachable from” the handle.

The present invention provides a technology for the safe, easy and inexpensive peeling of the skin that simultaneously causes a medicinal or cosmetical substance to penetrate the skin, doing so without subjecting the recipient to pain.

What is claimed is:

1. A skin-peeling pad comprising a plurality of non-needle shaped particles averaging ≦1 mm in size held in pad surface.

2. The skin-peeling pad in claim 1, where said non-needle shaped particles are adhered and held onto said pad surface by an adhesive or binder; and
where said adhesive or binder is selected from the group consisting of silicone, polyurethane, polyethylene, natural rubber or other continuously or discontinuously formed material; and
where said adhesive or binder further comprises having a JIS hardness of ≦30 degrees or a Shore hardness of ≦30 A, which is approximately equivalent to the hardness of the stratum corneum layer of the skin.

3. The skin-peeling pad in claim 2, where said non-needle shaped particles are made of a material selected from the group consisting of glass, quartz, sapphire, ceramic, polycarbonate, acrylic resin, PET (polyethylene terephthalate) or other hard plastic material having a degree of hardness sufficient to peel skin.

4. The skin-peeling pad in claim 3, where said non-needle shaped particles have a plurality of pores ≦1 micron in size; and where said non-needle shaped particles have a BET (Brunaure-Emmett-Teller value) specific surface area of ≧100 m²/gram.

5. The skin-peeling pad in claim 4, where said plurality of pores on said non-needle shaped particles contain a substance selected from the group consisting of medicinal or cosmetical; and where said substance is administered to the skin as it is peeled.

6. The skin-peeling pad in claim 1, where said non-needle shaped particles are made of a material selected from the group consisting of glass, quartz, sapphire, ceramic, polycarbonate, acrylic resin, PET (polyethylene terephthalate) or other hard plastic material having a degree of hardness sufficient to peel skin.

7. The skin-peeling pad in claim 6, where said non-needle shaped particles have a plurality of pores ≦1 micron in size; and where said non-needle shaped particles have a BET (Brunaure-Emmett-Teller value) specific surface area of ≧100 m²/gram.

8. The skin-peeling pad in claim 7, where said plurality of pores on said non-needle shaped particles contain a substance selected from the group consisting of medicinal or cosmetical; and where said substance is administered to the skin as it is peeled.

9. The skin-peeling pad in claim 1, where said non-needle shaped particles have a plurality of pores ≦1 micron in size; and where said non-needle shaped particles have a BET (Brunaure-Emmett-Teller value) specific surface area of ≧100 m²/gram.

10. The skin-peeling pad in claim 9, where said plurality of pores on said non-needle shaped particles contain a substance selected from the group consisting of medicinal or cosmetical; and where said substance is administered to the skin as it is peeled.

11. The skin-peeling pad in claim 2, where said non-needle shaped particles have a plurality of pores ≦1 micron in size; and where said non-needle shaped particles have a BET (Brunaure-Emmett-Teller value) specific surface area of ≦100 m²/gram.

12. The skin-peeling pad in claim 11, where said plurality of pores on said non-needle shaped particles contain a substance selected from the group consisting of medicinal or cosmetical; and where said substance is administered to the skin as it is peeled.

13. A holding device for said skin-peeling pad in claim 1.
